(19) United States
(12) Patent Application Publication
(10) Pub. No.: US 2010/0311642 A1
(43) Pub. Date: Dec. 9, 2010

(54) USE OF V2 RECEPTOR ANTAGONISTS IN COMBINATION WITH VASO PRESSINERGIC AGNOSTS

(75) Inventors: Pierre J-m.Riviere, San Diego, CA (US); Sudar Alagarsamy, San Diego, CA (US); Claudio Daniel Schteingart, San Diego, CA (US); Regent Laporte, San Diego, CA (US)

Correspondence Address: MERCHANT & GOULD PC, P.O. BOX 2903, MINNEAPOLIS, MN, 55402-0903, US

(73) Assignee: Ferring B.V., Hoofddorp (NL)

(21) Appl. No.: 12/680,452

(22) PCT Filed: Sep. 25, 2008
(86) PCT No.: PCT/US08/11097

§ 371 Date: Aug. 23, 2010

Related U.S. Application Data
(60) Provisional application No. 60/960,397, filed on Sep. 28, 2007.

(51) Int. Cl. A61K 38/11; A61K 31/55; A61K 31/5377; A61P 7/00; A61P 11/00

(52) U.S. Cl. 514/1.4; 514/215; 514/213.01; 514/235.2; 514/10.9; 514/1.5

(57) ABSTRACT

The combination of vasopressin V2 receptor antagonists and vasopressin receptor agonists (selective or non-selective) is described, including pharmaceutical compositions, including kits, for administering, and methods and uses of such a combination, e.g. for treatment of critical care diseases and conditions requiring control of arterial blood pressure or for treatment of hypotension. Also described is the use of a combination of a vasopressin V2 receptor antagonist with a vasopressin receptor agonist for the manufacture of a medicament for treatment of critical care conditions requiring control of arterial blood pressure, vascular leak and/or vasodilation, methods for treatment of a critical care condition in a mammal needing control of arterial blood pressure, vascular leak and/or vasodilation, the method comprising administering to the mammal a therapeutically effective amount of a vasopressin V2 receptor antagonist and administering to the mammal a therapeutically effective amount of a vasopressin receptor agonist, and a kit comprising a therapeutically effective amount of one or more vasopressin V2 receptor antagonists in a pharmaceutically acceptable composition and a therapeutically effective amount one or more vasopressin receptor agonists in a pharmaceutically acceptable composition.

### BACKGROUND

Peptidic vasopressin receptor agonists, such as terlipressin, have recently (see e.g. O'Brian et al., Lancet 359 (9313):1209-10, Jun. 4, 2002) received increased attention for clinical use in treatment of critical care diseases and conditions, including shock of hypovolemic (e.g. hemorrhagic) or vasodilatory (e.g. septic) origin, bleeding esophageal varices (BEV), hepatorenal syndrome (HRS), cardiopulmonary resuscitation and anesthesia-induced hypotension. They have also been shown to have clinical use in the treatment of orthostatic hypotension, paracentesis-induced circulatory dysfunction, intra-operative blood loss and blood loss associated with burn débridement and epistaxis.

The efficacy of current vasopressin receptor agonists in clinical use for treatment of critical care diseases and conditions is attributed to their activity at the V1a receptor. However, non-specific receptor agonist activity is the main disadvantage of these existing compounds, e.g. [Phe-2,Orn8]OT (See for Example, 1f in U.S. Pat. No. 3,352,843) and arginine-vasopressin (AVP). Agonist activity at one or more related receptors, such as V1b, V2 and/or oxytocin (OT) receptors, may potentially generate undesirable side effects and safety concerns. As an example, V2 receptor activation induces antidiuresis, releases of coagulation/thrombolysis factors, and induces vasodilation/hypotension with reflex tachycardia. Vasodilation/hypotension with reflex tachycardia has also been attributed to agonist activity/activation at the OT receptor.

The present invention provides for treatment of critical care diseases and conditions where the treatment has reduced or eliminated side effects related to non-specific receptor agonist activity of vasopressinergic receptor agonists.

### DESCRIPTION

The present invention relates to a combination of vasopressin V2 receptor antagonists and vasopressin receptor agonists (selective or non-selective), to pharmaceutical compositions, including kits, for administering, and to the methods and uses of such a combination, e.g. for treatment of critical care diseases and conditions requiring control of arterial blood pressure or for treatment of hypotension.

The present invention provides methods of use of a combination of one or more vasopressin receptor agonist compounds and one or more vasopressin V2 receptor antagonist compounds for treatment of conditions associated with critical care. The compounds of the combination may be concurrently or consecutively administered to a subject in need of treatment. In one embodiment, a vasopressin V2 receptor antagonist is administered before a vasopressin receptor agonist is administered to a subject in need of treatment. The time between administration of the vasopressin V2 receptor antagonist and the vasopressin receptor agonist during consecutive administration is between 1 second and 1 hour. In another embodiment, a vasopressin receptor agonist is administered to a subject in need of treatment before a vasopressin V2 receptor antagonist is administered. The time between consecutive administration of the vasopressin receptor agonist and administration of the vasopressin V2 receptor antagonist is between 1 second and 1 hour.

### Vasopressin V2 Receptor Antagonists

Suitable vasopressin V2 receptor antagonists for use as described herein are compounds which block the activity of or deactivate the V2 receptor. The vasopressin V2 receptor antagonists are either selective vasopressin V2 receptor antagonists or non-selective vasopressin V2 receptor antagonists. Suitable non-selective vasopressin V2 receptor antagonists are antagonists at vasopressin V2 receptors, but may also have antagonist activity at other related receptors, including V1A, V1B, OT or a combination thereof. In one embodiment, the non-selective vasopressin V2 receptor antagonist according to the present disclosure is conivaptan, and pharmaceutically acceptable salts and solvates thereof. In an embodiment, a non-selective vasopressin V2 receptor antagonist has greater antagonist activity at the vasopressin V2 receptor than at one or more related receptors. In a further embodiment, a non-selective vasopressin V2 receptor antagonist has greater antagonist activity at the vasopressin V2 receptor than at the V1a receptor. One method of measuring relative receptor antagonist activity is by in vitro receptor assays, described below.

In an embodiment, the vasopressin V2 receptor antagonists are selective vasopressin V2 receptor antagonists. Selective vasopressin V2 receptor antagonists are antagonists at vasopressin V2 receptors, but demonstrate no detectable or limited antagonist activity at the vasopressin V1a receptor.

In an embodiment, the selectivity of an antagonist compound for the V2 receptor compared to the V1a receptor is characterized by the relative Ki at the V2 receptor to the Ki at the V1a receptor. In certain of these embodiments, the ratio of the Ki values for V2:V1a is at least 10:1, may be at least 100:1, and may even be at least 1000:1.

In an embodiment, selective vasopressin V2 receptor antagonists have a Ki for the V2 receptor <100 nM. In a further embodiment, selective vasopressin V2 receptor antagonists have a Ki for the V2 receptor of <10 nM. In a still further embodiment, selective vasopressin V2 receptor antagonists have a Ki for the V2 receptor <1 nM.

In addition to the above embodiments, selective vasopressin V2 receptor antagonists have a Ki for the V1a receptor >100 nM. In further embodiments, Ki for the V1a receptor >500 nM, and in certain further embodiments, Ki for the V1a receptor >1000 nM.

Examples of suitable selective vasopressin V2 receptor antagonists include mozavaptan, tolvaptan, tolvaptan phosphate ester, satavaptan (SR-121463), lixivaptan, RWJ-351647, VP-343, VP-339, [Pmp1,D-Ile2,Ile4,Arg8,Ala9]vasopressin, [Pmp1,D-Ile2,Ile4,Arg8]vasopressin, [Pmp1,D-Ile2,Ile4,Arg8]vasopressin-(1-8)-OH, combinations thereof, and pharmaceutically acceptable salts and solvates thereof.

The chemical structures of conivaptan, mozavaptan, and lixivaptan are shown below. Lixivaptan may be synthesized according to: (a) Martinez-Castelao, (2001) A. Curr. Opin. Investig. Drugs 2:525; (b) Albright, et al. (1998) J. Med. Chem.41:2442; or (c) Albright, et al. (2000) Bioorg. Med. Chem. Lett., 10:695. Conivaptan may be synthesized according to: (a) Norman, et al. (2000) Drugs Fut. 2000, 25:1121; (b) Matsushita, et al. (2000) Chem. Pharm. Bull. 48:21. Mozavaptan may be synthesized according to: (a) Prous, et al. (1993) J. Drugs Fut. 18:802; (b) Ogawa et al. (1996) J. Med. Chem. 39:3547.

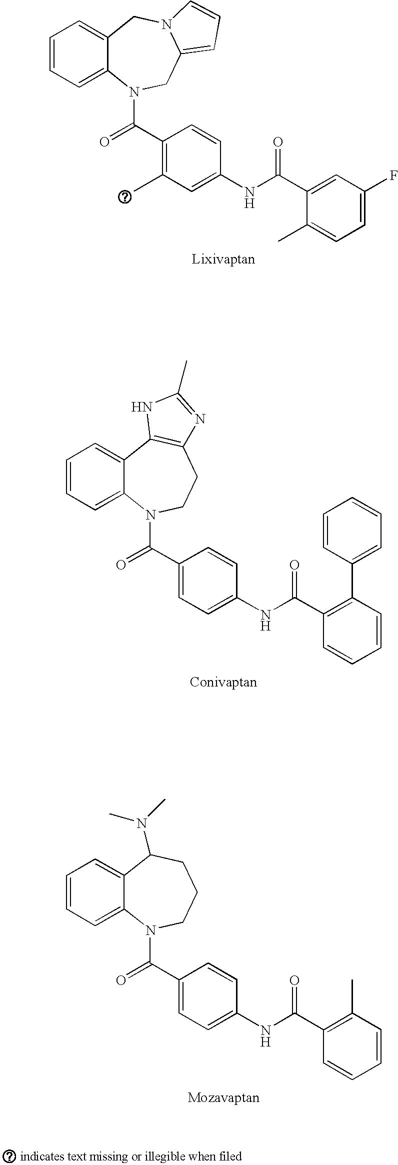

VP-343 is known chemically as ((N-4[[[(2S,3aR)-2-hydroxy-2,3,3a,4-tetrahydropyrrolo[1,2-a]quinoxalin-5(1H)-yl]carbonyl]phenyl]-4′-methyl[1,1′-biphenyl]-2-carboxamide]. VP-339 is known chemically as (N-[4-[[(11aS)-2,3,11,11a-tetrahydro-1H-pyrrolo[2,1-c][1,4]benzodiazepine-10(5H)-yl]carbonyl]phenyl]-[1,1′-biphenyl]-2-carboxamide). VP-343 and VP-339 may be synthesized according to the procedure of Ohtake et al. (1999) Bioorg. Med. Chem. 7:1247-1254.

RWJ-351647, also known as M0002, is a nonpeptide compound undergoing clinical trials. See Thuluvath et al. (2006) Aliment. Pharmacol. Ther. 24:973-982; Ros et al. (2005) Brit. J. Pharmacol. 146:654-661. RWJ-351647 may be synthesized according to Matthews et al. (2004) Bioorg. Med. Chem. Lett.82:2747-2752.

[Pmp1,D-Ile2,Ile4,Arg8,Ala9]vasopressin, [Pmp1,D-Ile2,Ile4,Arg8]vasopressin, [Pmp1,D-Ile2,Ile4,Arg8]vasopressin-(1-8)-OH, may be synthesized by known methods for solution phase or solid-phase synthesis of peptidic compounds.

### Vasopressin Receptor Agonists

Suitable vasopressin receptor agonists for use as described herein are compounds having non-selective agonist activity at the vasopressin receptors. Suitable vasopressin receptor agonists for use in the present invention include compounds that activate the vasopressin V1a receptor, but also may have agonist activity at one or more other related receptors, such as V1b, V2, or OT receptors. In various embodiments, suitable vasopressin receptor agonists have known agonist activity at the V1a receptor and V2 receptor.

In an embodiment, a non-selective vasopressin receptor agonist for use according to the present disclosure has greater activity at the vasopressin V1a receptor than at the vasopressin V2 receptor. In another embodiment, a non-selective vasopressin receptor agonist for use according to the present disclosure has greater activity at the vasopressin V2 receptor than at the vasopressin V1a receptor. One method of measuring relative receptor activities is by in vitro receptor assays, described below.

Examples of suitable non-selective vasopressin receptor agonists include arginine vasopressin (AVP), lysine vasopressin (LVP), terlipressin (G1y3-LVP), [Gly2]LVP, [Gly1]-LVP, felypressin, ornithine vasopressin (OVP), combinations thereof and pharmaceutically acceptable salts and solvates thereof.

### Combinations of Vasopressin V2 Receptor Antagonist and Vasopressin V1a Receptor Agonists

Preferred embodiments of combinations of vasopressin V2 receptor antagonist and non-selective vasopressin receptor agonist according to the present disclosure are presented in Table 1.

TABLE 1

| Vasopressin | |
|---|---|
| receptor agonist | V2 receptor antagonist |
| AVP | mozavaptan |
| AVP | tolvaptan |
| AVP | tolvaptan phosphate ester |
| AVP | satavaptan |
| AVP | lixivaptan |
| AVP | conivaptan |
| AVP | RWJ-351647 |
| AVP | VP-343 |
| AVP | VP-393 |
| AVP | [Pmp1, D-Ile2, Ile4, Arg8, Ala9]vasopressin, |
| AVP | [Pmp1, D-Ile2, Ile4, Arg8]vasopressin |
| AVP | [Pmp1, D-Ile2, Ile4, Arg8]vasopressin-(1-8)-OH |
| LVP | mozavaptan |
| LVP | tolvaptan |
| LVP | tolvaptan phosphate ester |
| LVP | satavaptan |
| LVP | lixivaptan |
| LVP | conivaptan |
| LVP | RWJ-351647 |
| LVP | VP-343 |
| LVP | VP-393 |
| LVP | [Pmp1, D-Ile2, Ile4, Arg8, Ala9]vasopressin, |
| LVP | [Pmp1, D-Ile2, Ile4, Arg8]vasopressin |
| LVP | [Pmp1, D-Ile2, Ile4, Arg8]vasopressin-(1-8)-OH |
| terlipressin | mozavaptan |
| terlipressin | tolvaptan |
| terlipressin | tolvaptan phosphate ester |
| terlipressin | satavaptan |
| terlipressin | lixivaptan |
| terlipressin | conivaptan |
| terlipressin | RWJ-351647 |
| terlipressin | VP-343 |
| terlipressin | VP-393 |
| terlipressin | [Pmp1, D-Ile2, Ile4, Arg8, Ala9]vasopressin, |
| terlipressin | [Pmp1, D-Ile2, Ile4, Arg8]vasopressin |
| terlipressin | [Pmp1, D-Ile2, Ile4, Arg8]vasopressin-(1-8)-OH |
| [Gly2]LVP | mozavaptan |
| [Gly2]LVP | tolvaptan |
| [Gly2]LVP | tolvaptan phosphate ester |
| [Gly2]LVP | satavaptan |
| [Gly2]LVP | lixivaptan |
| [Gly2]LVP | conivaptan |
| [Gly2]LVP | RWJ-351647 |
| [Gly2]LVP | VP-343 |
| [Gly2]LVP | VP-393 |
| [Gly2]LVP | [Pmp1, D-Ile2, Ile4, Arg8, Ala9]vasopressin, |
| [Gly2]LVP | [Pmp1, D-Ile2, Ile4, Arg8]vasopressin |
| [Gly2]LVP | [Pmp1, D-Ile2, Ile4, Arg8]vasopressin-(1-8)-OH |
| [Gly1]LVP | mozavaptan |
| [Gly1]LVP | tolvaptan |
| [Gly1]LVP | tolvaptan phosphate ester |
| [Gly1]LVP | satavaptan |
| [Gly1]LVP | lixivaptan |
| [Gly1]LVP | conivaptan |
| [Gly1]LVP | RWJ-351647 |
| [Gly1]LVP | VP-343 |
| [Gly1]LVP | VP-393 |
| [Gly1]LVP | [Pmp1, D-Ile2, Ile4, Arg8, Ala9]vasopressin, |
| [Gly1]LVP | [Pmp1, D-Ile2, Ile4, Arg8]vasopressin |
| [Gly1]LVP | [Pmp1, D-Ile2, Ile4, Arg8]vasopressin-(1-8)-OH |
| felypressin | mozavaptan |
| felypressin | tolvaptan |
| felypressin | tolvaptan phosphate ester |
| felypressin | satavaptan |
| felypressin | lixivaptan |
| felypressin | conivaptan |
| felypressin | RWJ-351647 |
| felypressin | VP-343 |
| felypressin | VP-393 |
| felypressin | [Pmp1, D-Ile2, Ile4, Arg8, Ala9]vasopressin, |
| felypressin | [Pmp1, D-Ile2, Ile4, Arg8]vasopressin |
| felypressin | [Pmp1, D-Ile2, Ile4, Arg8]vasopressin-(1-8)-OH |
| OVP | mozavaptan |
| OVP | tolvaptan |
| OVP | tolvaptan phosphate ester |
| OVP | satavaptan |
| OVP | lixivaptan |
| OVP | conivaptan |
| OVP | RWJ-351647 |
| OVP | VP-343 |
| OVP | VP-393 |
| OVP | [Pmp1, D-Ile2, Ile4, Arg8, Ala9]vasopressin, |
| OVP | [Pmp1, D-Ile2, Ile4, Arg8]vasopressin |
| OVP | [Pmp1, D-Ile2, Ile4, Arg8]vasopressin-(1-8)-OH |

The combinations presented above encompass pharmaceutically acceptable salts and solvates of the above compounds. In further embodiments, a composition, kit, use or method according to the present disclosure includes one or more of the above combinations of compounds.

### Pharmaceutical Compositions and Administration

For use in methods of the present disclosure, one or more vasopressin V2 receptor antagonists and one or more vasopressin receptor agonists are present in one or more pharmaceutical compositions. In an embodiment, one pharmaceutical composition includes one or more vasopressin V2 receptor antagonists and one or more vasopressin receptor agonists. In a further embodiment, one pharmaceutical composition includes a vasopressin V2 receptor antagonist and a vasopressin receptor agonist.

Alternatively, one or more vasopressin V2 receptor antagonists are contained in one or more separate pharmaceutical compositions and one or more vasopressin receptor agonists are contained in one or more separate other pharmaceutical compositions. Where the vasopressin V2 receptor antagonists and the vasopressin receptor agonists are contained in one or more separate other pharmaceutical compositions, the pharmaceutical compositions may be administered separately or mixed prior to administration. Where the vasopressin V2 receptor antagonists and the vasopressin receptor agonists are contained in one or more separate other pharmaceutical compositions, the compositions may be provided in a kit.

The one or more pharmaceutical compositions used when practising the present disclosure may be adapted for oral, intravenous, topical, intraperitoneal, nasal, buccal, sublingual or subcutaneous administration or for administration via the respiratory tract e.g. in the form of an aerosol or an air-suspended fine powder. The one or more pharmaceutical compositions may thus for instance be in the form of tablets, capsules, powders, microparticles, granules, syrups, suspensions, solutions, transdermal patches or suppositories.

The one or more pharmaceutical compositions used may optionally comprise e.g. at least one further additive selected from a disintegrating agent, binder, lubricant, flavoring agent, preservative, colorant and any mixture thereof. Examples of such and other additives are found in “Handbook of Pharmaceutical Excipients”; Ed. A. H. Kibbe, 3^{rd} Ed., American Pharmaceutical Association, USA and Pharmaceutical Press UK, 2000.

The one or more pharmaceutical compositions used are most preferably adapted for parenteral administration. The one or more pharmaceutical compositions each may comprise a sterile aqueous preparation of one or more of the compounds of the disclosure. The one or more pharmaceutical compositions are generally isotonic with the blood of the recipient. The sterile aqueous preparation may be formulated according to known methods using suitable dispersing or wetting agents and suspending agents. The injectable aqueous formulation Remestyp® (terlipressin) is exemplary of a suitable pharmaceutical formulation type. The preparation may also be a sterile injectable solution or suspension in a diluent or solvent, for example as a solution in 1,3-butane diol. Water, Ringer's solution, and isotonic sodium chloride solution are exemplary acceptable diluents. Sterile, fixed oils may be employed as a solvent or suspending medium. Bland fixed oils, including synthetic mono or di-glycerides, and fatty acids, such as oleic acid, may also be used.

The typical dosage of the compounds used according to the present disclosure varies within a wide range and will depend on various factors such as the individual needs of each patient and the route of administration. As part of the combination of the present disclosure, the dosage of the non-selective Via agonist administered by infusion is generally within the range of 0.005-100 μg/kg body weight per hour. Frequently, the dosage of the non-selective V1a agonist administered by infusion is within the range of 0.005-10 μg/kg body weight per hour. Often, the dosage of the non-selective V1a agonist administered by infusion is within the range of 0.01-1 μg/kg body weight per hour. On occasion, the dosage of the non-selective V1a agonist administered by infusion is within the range of 0.01-0.1 μg/kg body weight/h, or even may be within the range of 0.02-0.1 μg/kg body weight/h. The dosage of the non-selective V1a agonist administered by i.v. bolus is generally within the range of 0.01-200 μg/kg body weight. Typically, the dosage of the non-selective V1a agonist administered by bolus is within the range of 0.1 to 100 μg/kg body weight. Often, the dosage of the non-selective V1a agonist administered by bolus is within the range of 0.5 to 10 μg/kg body weight, on occasion within the range of 0.5-2 μg/kg body weight, or even maybe in the range of 1-1.5 μg/kg body weight. A physician of ordinary skill in the art will be able to optimize the dosage to the situation at hand.

As part of the combination of the present disclosure, the dosage of the selective V2 antagonist administered is an amount sufficient to reduce or counteract the V2 agonist activity of the administered non-selective V1a agonist on the vasopressin V2 receptor. The dosage of the selective V2 antagonist administered by infusion is generally within the range of 1-300 μg/kg body weight per hour. Typically, the dosage of the selective V2 antagonist administered by infusion is generally within the range of 10-250 μg/kg body weight per hour. A physician of ordinary skill in the art will be able to optimize the dosage to the situation at hand.

### Critical Care Diseases and Conditions

Subjects in need of treatment are mammals, including but not limited to mice, rats, cats, dogs, sheep, goats, cows, horses, monkeys, apes and humans, which are in need of treatment for one or more critical care diseases or conditions indicated below. Critical care diseases and conditions which may be treated by a combination of vasopressin V2 receptor antagonist in combination with a vasopressin V1a agonist include those diseases and conditions wherein the patient is hypotensive or otherwise is in need of medical intervention to control arterial blood pressure, vascular leak and/or vasodilation.

Critical care diseases and conditions which may be treated by a combination of vasopressin V2 receptor antagonist in combination with a vasopressin V1a agonist include, but are not limited to, hypertensive gastropathy bleeding, sepsis, severe sepsis, septic shock, prolonged and severe hypotension, intradialytic hypotension, cardiac arrest, trauma related blood loss, vasodilatory shock induced by cardiopulmonary bypass, milrinone-induced vasodilatory shock in congestive heart failure, late phase hemorrhagic shock, hepatorenal syndrome type I, cardiovascular instability induced by brain death or anaphylactic shock, hypotension in severe sepsis, acute respiratory distress syndrome (ARDS) or acute lung injury (ALI), inadequate tissue oxygenation, e.g. stemming from nitrogen intoxication (hypoxic lactic acidosis) or carbon monoxide intoxication, shock induced by metformin intoxication, mitochondrial disease or cyanide poisoning, vascular leak syndrome (VLS) induced by interleukin-2 (IL-2) or other cytokines, denileukin diftitox or other immunotoxins, or ovarian hyperstimulation syndrome (OHSS), hypertension induced by end-stage renal disease (ESRD), severe burns, thermal injury, irritable bowel disease (IBD), including Crohn's disease and ulcerative colitis, reperfusion injury (e.g. stemming from thrombotic stroke, coronary thrombosis, cardio-pulmonary bypass, coronary artery bypass graft, limb or digit replantation, organ transplantation, bypass enteritis, bypass arthritis, thermal injury, crush injury/compartment syndrome), infant respiratory distress syndrome (IRDS, RDS), severe acute respiratory syndrome (SARS), ascites, vasodepressor syncope, e.g. vasovagal syncope, postural hypotension with syncope or neurocardiogenic syncope, toxic shock syndrome, and idiopathic systemic capillary leak syndrome (Clarkson's disease).

For more detail on the above indications and conditions see e.g. the references Bruha, R. et al. Hepatogastroenterology 49:1161-1166, 2002; Landry, D. W. et al. Circulation 95:1122-1125, 1997; Argenziano, M. et al. Circulation 96:11-286-11-290, 1997; Landry, D. W. et al. U.S. patent application published as no. 2004-229798; Wenzel, V. et al. N. Engl. J. Med. 350:105-113, 2004; Okin, C. R. et al. Obstet. Gynecol. 97:867-872, 2001; Gold, J. et al. Am. J. Cardiol. 85:506-508, 2000; Sharma, R. M. and Setlur, R. Anest. Analg. 101:833-834, 2005; Solanik, P. et al. J. Gastroenterol. Hepatol. 18:152-156, 2000; Yoshioka, T. et al. Neurosurgery 18:565-567, 1986; Kill, C. et al. Int. Arch. Allergy Immunol. 134:260-261, 2004; Westphal, M. et al. Annual Congress of the Society of Critical Care Medicine, Abstract no. 196470, 2006; Landry, D. W. and Oliver, J. A. N. Engl. J. Med. 345(8):588-595, 2001; Baluna, R. and Vitetta, E. S. Immunopharm. 37:117-132, 1997; Delbaere, A. et al. Endocrine. 26:285-290, 2005; Agarwal, R. Cardiol. Clin. 23:237-248, 2005; Demling, R. H. J. Burn Care Rehabil. 26:207-227, 2005; Bonder, C. S, and Kubes, P. Am. J. Physiol. 284:729-733, 2003; Seal, J. B. and Gewertz, B. L. Ann. Vasc. Surg. 19:572-584, 2005; Zoban, P., Cerny, M. Physiol. Res. 52:507-516, 2003; Bermejo, J. F. and Munoz-Fernandez, M. A. Viral Immunol. 17:535-544, 2004; Arroyo, V. Ann. Hepatol. 1:72-79, 2002; Hainsworth, R. Clin. Auton. Res. 14 Suppl 1:18-24, 2004; Chuang, Y. Y. et al. Paediatr. Drugs. 7:11-25, 2005; Cau, C. Minerva Med. 90:391-396, 1999.

### Methods and Uses

In a first embodiment, the invention relates to a method for treatment of hypertensive gastropathy bleeding, sepsis, severe sepsis, septic shock, prolonged and severe hypotension, intradialytic hypotension, cardiac arrest, trauma related blood loss, vasodilatory shock induced by cardio-pulmonary bypass, milrinone-induced vasodilatory shock in congestive heart failure, hepatorenal syndrome type I, anaphylactic shock, or cardiovascular instability induced by brain death, wherein said method comprises administering to a mammal, in need of treatment there for, of a therapeutically effective amount of the combination of vasopressin V2 receptor antagonist and vasopressin receptor agonist.

In a second embodiment, the invention relates to a method for treatment of hypotension in severe sepsis, acute respiratory distress syndrome or acute lung injury, wherein said method comprises administering to a mammal, in need of treatment there for, of a therapeutically effective amount of the combination of vasopressin V2 receptor antagonist and vasopressin receptor agonist.

In a third embodiment, the invention relates to a method for treatment of inadequate tissue oxygenation, shock induced by metformin intoxication, mitochondrial disease or cyanide poisoning, vascular leak syndrome induced by interleukin-2 or other cytokines, denileukin diftitox or other immunotoxins, or ovarian hyperstimulation syndrome, hypertension induced by end-stage renal disease, severe burns, thermal injury, irritable bowel disease, ulcerative colitis, reperfusion injury, infant respiratory distress syndrome, severe acute respiratory syndrome, ascites, vasodepressor syncope, including vasovagal syncope, postural hypotension with syncope or neurocardiogenic syncope, toxic shock syndrome, idiopathic systemic capillary leak syndrome (Clarkson's disease), wherein said method comprises administering to a mammal, in need of treatment there for, of a therapeutically effective amount of the combination of vasopressin V2 receptor antagonist and vasopressin receptor agonist.

Use of the combination of vasopressin V2 receptor antagonist and vasopressin receptor agonist are for the manufacture of a medicament for treatment of conditions as defined in the first embodiment supra.

Further uses of the combination of vasopressin V2 receptor antagonist and vasopressin receptor agonist are for the manufacture of a medicament for treatment of hypotension in severe sepsis, acute respiratory distress syndrome (ARDS) or acute lung injury (ALI).

Still further uses of the combination of vasopressin V2 receptor antagonist and vasopressin receptor agonist are for the manufacture of a medicament for treatment of conditions as defined in the third embodiment supra.

### EXPERIMENTAL

### Biological Testing

### I. Sheep Sepsis Data

A. Description of Model & Experimental Design
Septic shock is caused by hypotension secondary to vasodilation. This hypotension is resistant to fluid resuscitation and requires the use of vasopressor agents.
In a sheep model of sepsis, we observed a difference between treatment groups receiving: (i) AVP, a non-selective V1a/V2 receptor agonist that has been shown to effectively treat this hypotension in sepsis patients, or (ii) a selective nonapeptidic V1a receptor agonist. Both treatments were efficacious in maintaining mean arterial pressure (MAP); however, AVP-treated animals showed symptoms of vascular leak syndrome, while animals treated with the selective V1a receptor agonist did not exhibit symptoms of vascular leak.
We hypothesized that the difference between AVP and the selective V1a receptor agonist on vascular leak syndrome is due to the V2 receptor agonist activity of AVP.
To test this hypothesis, a selective V2 receptor agonist, desmopressin, was co-administered together with the selective V1a receptor agonist by constant infusion in a sheep model of sepsis, starting from the time of injury. The combination of a selective V2 receptor agonist, desmopressin, and a selective V1a receptor agonist models to some extent the non-selective V1a/V2 receptor agonist activity of vasopressin and other related compounds having non-selective activity.

Method: Sheep are surgically instrumented ahead of the study. After obtaining baseline data, they are anesthetized and insufflated with 48 breaths of cotton smoke and 10¹¹ colony forming units of Pseudomonas aeruginosa instilled into their airways via a tracheostomy. The sheep are then placed on a ventilator and awakened for the study. They are resuscitated with Ringer's solution to maintain left atrial and central venous pressures and hematocrit at baseline levels. If, despite fluid management, mean arterial pressure (MAP) falls by 10 mm Hg from baseline, a continuous intravenous infusion of AVP, the selective V1a receptor agonist, or the selective V1a receptor agonist+the selective V2 agonist desmopressin is initiated and titrated to keep MAP within this limit, except in a septic control group and in a non-septic sham group. Parameters measured include Mean Arterial Pressure (a measure of vasodilation/hypotension) and Fluid Balance (a measure of vascular leak).

B. Data and Analysis:

V1a agonist vs. AVP vs. V1a agonist+V2 agonist; effects on vascular leak and mean arterial pressure (MAP).

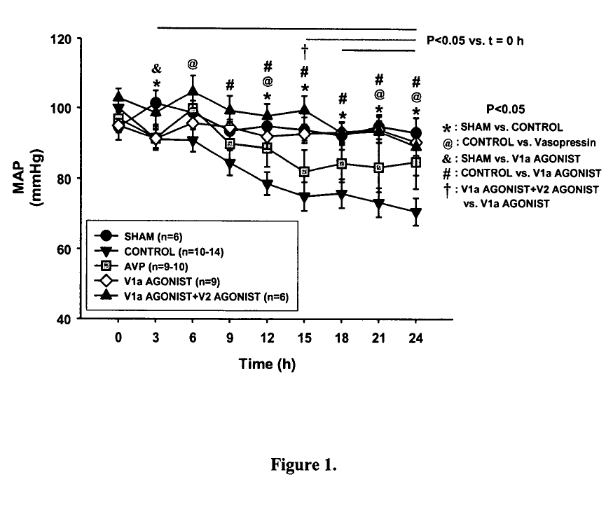

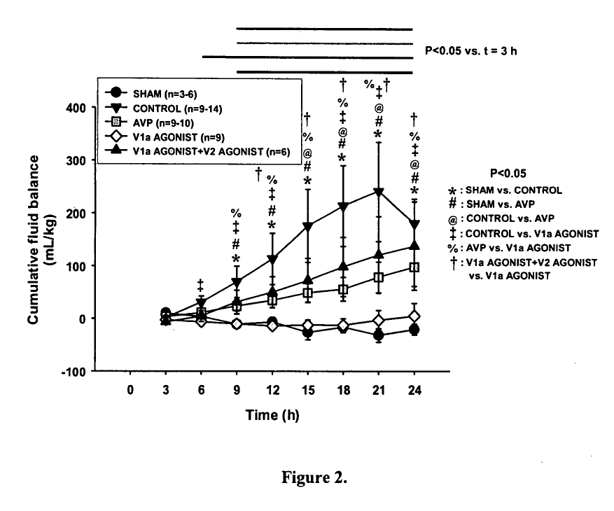

Maintained levels of mean arterial pressure (MAP) measured in mmHg over 24 hours in the sheep test subjects is presented in FIG. 1. Cumulative fluid levels for the sheep test subjects are presented in FIG. 2.

The V1a agonist alone was effective in raising MAP, while minimizing fluid retention. AVP was less effective in both cases. Adding a V2 agonist to the V1a agonist decreased the effectiveness of the V1a agonist to the level of AVP.

Additional information about sepsis model and method available in Murakami K, Bjertnaes L T, Schmalstieg F C, McGuire R, Cox R A, Hawkins H K, Herndon D N, Traber L D, Traber D L (2002): A novel animal model of sepsis after acute lung injury in sheep. Crit. Care Med, 30:2083-2090.

### II. Rat Mesenteric Post-Capillary Venule Permeability Data

A. Description of Model & Experimental Design

We tested the hypothesis that the selective V2 receptor agonist desmopressin would potentiate the increase in microvessel permeability induced by platelet activating factor [PAF], an inflammatory mediator involved in sepsis.

Method: Individual venular microvessels in rat mesentery were cannulated and perfused with mammalian Ringer containing 10 mg/ml albumin to measure hydraulic conductivity (Lp) by the modified Landis technique. The vessels were then exposed to PAF (10 nM) with monitoring of hydraulic conductivity (Lp). The same vessels are then exposed to a selective V2 receptor agonist, desmopressin, followed by a second exposure to PAF with monitoring of hydraulic conductivity (Lp).

B. Data and Analysis:

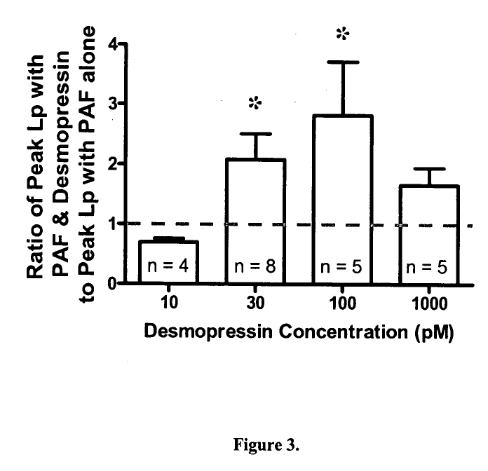

On average, Lp increased to a peak value in 5 minutes then returned toward control values after first exposure to PAF (10 nM). The same vessels were then exposed to desmopressin at concentrations of 10, 30, 100 pM for 40 minutes, followed by a second exposure to PAF. Measurements hydraulic conductivity (Lp) following second exposure to PAF are shown in FIG. 3. The second exposure to PAF in the presence of desmopressin produced a significant increase in the peak Lp for desmopressin concentrations of 30 pM (2.1 fold±0.4 SEM, n=8) and 100 pM (2.8 fold±0.9 SEM, n=5) relative to the peak Lp with PAF alone. In contrast, the peak Lp with PAF and 10 pM desmopressin was not increased relative to the peak Lp with PAF alone. In control experiments, a second exposure to PAF alone did not show an increase in peak Lp relative to the first exposure to PAF. Thus the potentiation of the PAF response by desmopressin occurred only at supra-antidiuretic (>10 pM) concentrations.

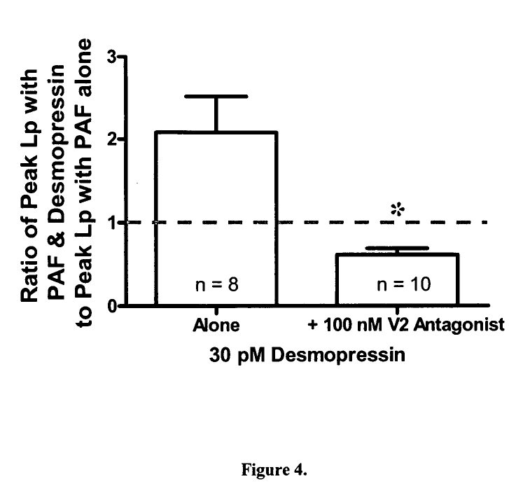

In an additional 10 experiments, a V2 receptor antagonist (100 nM; Ferring) abolished the 30 pM desmopressin potentiated increase in peak Lp with PAF. Data represented in bar graph of FIG. 4, which shows the ratio of Peak LP with PAF & Desmopressin to Peak Lp with PAF alone. These results conform to the hypothesis that supra-antidiuretic concentrations of V2 receptor agonists may contribute to sepsis induced increases in vascular leakage via a V2 receptor specific pathway.

Additional information about microvascular permeability model and method available in Curry, FE, “The Measurement of Hydraulic Conductivity”, Microcirculatory Technology, Academic Press, 1986, pp 429-446.

III. In vitro data—Reporter Gene Assays

A. Description of Model & Experimental Design

Agonist Reporter Gene Assays

Agonist activity of compounds on the human V1a receptor (hV1aR) and the rat V1a receptor (rV 1 aR) was determined in a transcriptional reporter gene assay in which HEK-293 cells were transiently co-transfected with either the human or rat V1a receptor, and a reporter DNA containing intracellular calcium responsive promoter elements regulating expression of firefly luciferase. See Boss, V., Talpade, D. J., Murphy, T. J. J. Biol. Chem. 1996, May 3; 271(18), 10429-10432 for further guidance on this assay. Cells were exposed to serial dilutions of compounds diluted 10-fold per dose for 5 hours, followed by lysis of cells, determination of luciferase activity, and determination of compound efficacies and EC50 values through non-linear regression.

Agonist activity of compounds on the human V2 receptor (hV2R) and the rat V2 receptor (rV2R) was determined in a transcriptional reporter gene assay in which HEK-293 cells were transiently transfected with either the human or rat V2 receptor, and a reporter DNA containing cyclic-AMP responsive promoter elements regulating expression of firefly luciferase. See Castarion, M. J., Spevak, W., Biochem Biophys Res Commun.1994 Jan. 28; 198(2):626-31 for further guidance on this assay. Cells were exposed to serial dilutions of compounds diluted 10-fold per dose for 5 hours, followed by lysis of cells, determination of luciferase activity, and determination of compound efficacies and EC50 values through non-linear regression.

Antagonist Reporter Gene Assays

Antagonist activity of compounds on the human V1a receptor (hV1aR) and the rat V1a receptor (rV 1 aR) was determined in a transcriptional reporter gene assay as in section III.A; see Boss, V. et al. supra. Cells were exposed to agonist (AVP) and serial dilutions of antagonist compounds diluted 10-fold per dose for 5 hours, followed by lysis of cells, determination of luciferase activity, and determination of compound efficacies and IC50 values through non-linear regression. Ki values were calculated from IC50 values.

Antagonist activity of compounds on the human V2 receptor (hV2R) and the rat V2 receptor (rV2R) was determined in a transcriptional reporter gene assay as in section III.A; see Castañón, M. J. et al. supra. Cells were exposed to agonist (AVP) and serial dilutions of antagonist compounds diluted 10-fold per dose for 5 hours, followed by lysis of cells, determination of luciferase activity, and determination of compound efficacies and IC50 values through non-linear regression. Ki values were calculated from IC50 values.

Results for transcriptional reporter gene assays are presented in Tables 2-5.

B. Data

TABLE 2
Characterization of Antagonist Compounds in Reporter Gene Assays

| | hV2 | | | hV1a | | |
|---|---|---|---|---|---|---|
| | Geomean | Efficacy | | Geomean | Efficacy | |
| Object Name | Ki (nM) | Antag (%) | N | Ki (nM) | Antag (%) | N |
| conivaptan | 3.2 | 92 | 4 | 27 | 81 | 7 |
| mozavaptan | 64.9 | 107 | 4 | >950 | 85 | 6 |
| satavaptan | 1.5 | 109 | 8 | >950 | 10 | 18 |
| tolvaptan | 2.7 | 97 | 4 | 109 | 101 | 8 |
| [Pmp1 DIle2 | 136 | 81 | 8 | 156 | 94 | 4 |
| Ile4 Ala9]AVP | | | | | | |
| [Pmp1, D- | 270 | 72 | 8 | 163.15 | 120 | 8 |
| Ile2, Ile4]AVP | | | | | | |
| [Pmp1, D- | >909 | 25 | 7 | >950 | 43 | 12 |
| Ile2, Ile4]AVP- | | | | | | |
| (1-8)-OH | | | | | | |

TABLE 3
Characterization of Antagonist Compounds in Reporter Gene Assays.

| | rV2 | | | rV1a | | |
|---|---|---|---|---|---|---|
| | Geomean | Efficacy | | Geomean | Efficacy | |
| Object Name | Ki (nM) | Antag (%) | N | Ki (nM) | Antag (%) | N |
| conivaptan | 0.19 | 97 | 4 | 2.8 | 99 | 6 |
| mozavaptan | 13 | 85 | 6 | >338 | 40 | 4 |
| satavaptan | 0.6 | 100 | 8 | >338 | 6 | 13 |
| tolvaptan | 3.3 | 102 | 6 | >338 | 26 | 8 |
| [Pmp1 DIle2 | 4.8 | 99 | 8 | >654 | 15 | 14 |
| Ile4 | | | | | | |
| Ala9]AVP | | | | | | |
| [Pmp1, D- | 10 | 97 | 2 | >338 | 26 | 8 |
| Ile2, Ile4]AVP | | | | | | |
| [Pmp1, D- | 36 | 105 | 4 | >338 | 26 | 7 |
| Ile2, Ile4]AVP- | | | | | | |
| (1-8)-OH | | | | | | |

TABLE 4
Characterization of Agonist Compounds in Reporter Gene Assays

| | hV2 | | | hV1a | | |
|---|---|---|---|---|---|---|
| | EC50 | Efficacy | | EC50 | Efficacy | |
| Object Name | (nM) | (%) | n | (nM) | (%) | n |
| [Orn8] | 0.45 | 97 | 11 | 0.69 | 99 | 7 |
| Vasopressin, OVP | | | | | | |
| H-Gly2-LVP | 284.06 | 66 | 4 | 191.19 | 49 | 9 |
| H-Gly-LVP | 20.33 | 96 | 10 | 89.44 | 87 | 6 |
| Lysine | 0.28 | 92 | 13 | 0.94 | 98 | 7 |
| Vasopressin, LVP | | | | | | |
| Arginine | 0.04 | 100 | 54 | 0.20 | 100 | 628 |
| vasopressin, AVP | | | | | | |
| H-Gly3-LVP, | 121.00 | 79 | 4 | 124.00 | 66 | 13 |
| terlipressin | | | | | | |

TABLE 5
Characterization of Agonist Compounds in Reporter Gene Assays

| | rV2 | | | rV1a | | |
|---|---|---|---|---|---|---|
| | EC50 | Efficacy | | EC50 | Efficacy | |
| Object Name | (nm) | (%) | n | (nM) | (%) | n |
| [Orn8] | 0.37 | 111 | 7 | 0.35 | 101 | 4 |
| Vasopressin, OVP | | | | | | |
| H-Gly2-LVP | 140.01 | 88 | 4 | 103.16 | 77 | 3 |
| H-Gly-LVP | 20.04 | 106 | 5 | 29.76 | 99 | 4 |
| Lysine | 1.11 | 99 | 8 | 0.20 | 99 | 4 |
| Vasopressin, LVP | | | | | | |
| Arginine | 0.03 | 100 | 62 | 0.07 | 100 | 282 |
| vasopressin, AVP | | | | | | |

Potency of receptor activation, as may be determined by in vitro reporter gene assays, is an important component of in vivo compound potency. The in vitro V1a receptor activity and the in vitro V2 receptor activity, in a mammal, of a non-selective V1a agonist is used to select one or more non-selective V1a agonists for use in the method of the present invention. The in vitro V2 receptor activity, in a mammal, of a selective V2 antagonist is used to select one or more selective V2 antagonists for use in the method of the present invention.

All references herein are indicative of the level of ordinary skill in the art to which this invention pertains and are incorporated herein by reference in their entireties.

1-14. (canceled)

15. A method for treatment of a critical care condition in a mammal needing control of arterial blood pressure, vascular leak and/or vasodilation, the method comprising: administering to the mammal a therapeutically effective amount of a vasopressin V2 receptor antagonist; and administering to the mammal a therapeutically effective amount of a vasopressin receptor agonist.

16. The method of claim 15, wherein the vasopressin V2 receptor antagonist is a selective vasopressin V2 receptor antagonist.

17. The method of claim 15, wherein the selective vasopressin V2 receptor antagonist comprises mozavaptan, tolvaptan, tolvaptan phosphate ester, satavaptan (SR-121463), lixivaptan, RWJ-351647, VP-343, VP-339, [Pmp1,D-112,Ile4,Arg8,Ala9]Vasopressin, [Pmp1,D-Ile2,Ile4,Arg8]Vasopressin, and [Pmp1,D-Ile2, Ile4,Arg8]Vasopressin-(1-8)-OH, combinations thereof, or pharmaceutically acceptable salts and solvates thereof.

18. The method of claim 15, wherein the vasopressin V2 receptor antagonist is a non-selective vasopressin V2 receptor antagonist.

19. The method of claim 18, wherein the non-selective vasopressin V2 receptor antagonist is conivaptan, or pharmaceutically acceptable salts and solvates thereof.

20. The method of claim 15, wherein the vasopressin receptor agonist comprises arginine vasopressin (AVP), lysine vasopressin (LVP), terlipressin (Gly3-LVP), [Gly2]LVP, [Gly1]-LVP., felypressin, ornithine vasopressin (OVP), combinations thereof, or pharmaceutically acceptable salts thereof.

21. (canceled)

22. The method of claim 15, wherein the vasopressin V2 receptor antagonist and vasopressin receptor agonist are administered concurrently.

23. The method of claim 15, wherein the vasopressin V2 receptor antagonist and the vasopressin receptor agonist are administered sequentially.

24. The method of claim 23, wherein the vasopressin V2 receptor antagonist is administered before said vasopressin receptor agonist and the time between administration thereof is between 1 second and 1 hour.

25. The method of claim 23, wherein the vasopressin receptor agonist is administered before said vasopressin V2 receptor antagonist and the time between administration thereof is between 1 second and 1 hour.

26. The method of claim 15, wherein the condition comprises hypertensive gastropathy bleeding, sepsis, severe sepsis, septic shock, prolonged and severe hypotension, intradialytic hypotension, cardiac arrest, trauma related blood loss, vasodilatory shock induced by cardio-pulmonary bypass, milrinone-induced vasodilatory shock in congestive heart failure, hepatorenal syndrome type I, anaphylactic shock, or cardiovascular instability induced by brain death.

27. The method of claim 15, wherein the condition comprises hypotension in severe sepsis, acute respiratory distress syndrome, or acute lung injury.

28. The method of claim 15, wherein the condition comprises inadequate tissue oxygenation, shock induced by metformin intoxication, mitochondrial disease or cyanide poisoning, vascular leak syndrome induced by interleukin-2 or other cytokines, denileukin diftitox or other immunotoxins, or ovarian hyperstimulation syndrome, hypertension induced by end-stage renal disease, severe burns, thermal injury, irritable bowel disease, reperfusion injury, infant respiratory distress syndrome, severe acute respiratory syndrome, ascites, vasodepressor syncope, including vasovagal syncope, postural hypotension with syncope or neurocardiogenic syncope, toxic shock syndrome, idiopathic systemic capillary leak syndrome (Clarkson's disease).

29. A composition comprising a vasopressin V2 receptor antagonist, a vasopressin receptor agonist, and one or more pharmaceutically acceptable excipients.

30. The composition of claim 29, wherein the vasopressin V2 receptor antagonist is a selective vasopressin V2 receptor antagonist.

31. The composition of claim 29, wherein the selective vasopressin V2 receptor antagonist comprises mozavaptan, tolvaptan, tolvaptan phosphate ester, satavaptan (SR-121463), lixivaptan, RWJ-351647., VP-343, VP-339, [Pmp1,D-I12,Ile4,Arg8,Ala9]Vasopressin, [Pmp1,D-Ile2,Ile4,Arg8]Vasopressin, and [Pmp1,D-Ile2, Ile4,Arg8]Vasopressin-(1-8)-OH, combinations thereof, or pharmaceutically acceptable salts and solvates thereof.

32. The composition of claim 29, wherein the vasopressin V2 receptor antagonist is a non-selective vasopressin V2 receptor antagonist.

33. The composition of claim 32 wherein the non-selective vasopressin V2 receptor antagonist comprises conivaptan, or pharmaceutically acceptable salts and solvates thereof.

34. The composition of claim 29, wherein the vasopressin receptor agonist comprises arginine vasopressin (AVP), lysine vasopressin (LVP), terlipressin (Gly3-LVP), [Gly2]LVP, [Gly1]-LVP, felypressin, ornithine vasopressin (OVP), combinations thereof, or pharmaceutically acceptable salts thereof.

35. (canceled)

36. The composition of claim 19, wherein the composition is injectable.

37. The composition of claim 29, wherein the composition is effective for treatment of a critical care condition in a mammal needing control of arterial blood pressure, vascular leak and/or vasodilation.

38. A kit comprising a therapeutically effective amount of one or more vasopressin V2 receptor antagonists in a pharmaceutically acceptable composition and a therapeutically effective amount one or more vasopressin receptor agonists in a pharmaceutically acceptable composition.

39. The kit of claim 38, wherein the vasopressin V2 receptor antagonist is a selective vasopressin V2 receptor antagonist.

40. The kit of claim 39, wherein the selective vasopressin V2 receptor antagonist comprises mozavaptan, tolvaptan, tolvaptan phosphate ester, satavaptan (SR-121463), lixivaptan, RWJ-351647, VP-343, VP-339, [Pmp1,D-I12,Ile4,Arg8,Ala9]Vasopressin, [Pmp1,D-Ile2,Ile4,Arg8]Vasopressin, and [Pmp1,D-Ile2, Ile4,Arg8]Vasopressin-(1-8)-OH, combinations thereof, or pharmaceutically acceptable salts and solvates thereof.

41. The kit of claim 38, wherein the vasopressin V2 receptor antagonist is a non-selective vasopressin V2 receptor antagonist.

42. The kit of claim 41 wherein the non-selective vasopressin V2 receptor antagonist is conivaptan, and pharmaceutically acceptable salts and solvates thereof.

43. The kit of claim 18, wherein the vasopressin receptor agonist comprises arginine vasopressin (AVP), lysine vasopressin (LVP), terlipressin (Gly3-LVP), [Gly2]LVP, [Gly1]-LVP, felypressin, ornithine vasopressin (OVP), combinations thereof, or pharmaceutically acceptable salts thereof.

44. (canceled)

45. The kit of claim 38, wherein said pharmaceutically acceptable compositions are administrable by injection.

46. The method of claims 15, wherein the combination of the vasopressin V2 receptor antagonist with the vasopressin receptor agonist comprises one of the following pairs:

| vasopressin | |
|---|---|
| receptor | |
| agonist: | vasopressin V2 receptor antagonist: |
| AVP | mozavaptan |
| AVP | tolvaptan |
| AVP | tolvaptan phosphate ester |
| AVP | satavaptan |
| AVP | lixivaptan |
| AVP | conivaptan |
| AVP | RWJ-351647 |
| AVP | VP-343 |
| AVP | VP-393 |
| AVP | [Pmp1, D-Ile4, Arg8, Ala9]vasopressin, |
| AVP | [Pmp1, D-Ile4, Arg8, Ala8]vasopressin |
| AVP | [Pmp1, D-Ile4, Arg8, Ala8]vasopressin-(1-8)-OH |
| LVP | mozavaptan |
| LVP | tolvaptan |
| LVP | tolvaptan phosphate ester |
| LVP | satavaptan |
| LVP | lixivaptan |
| LVP | conivaptan |
| LVP | RWJ-351647 |
| LVP | VP-343 |
| LVP | VP-393 |
| LVP | [Pmp1, D-Ile4, Arg8, Ala9]vasopressin, |
| LVP | [Pmp1, D-Ile4, Arg8, Ala8]vasopressin |
| LVP | [Pmp1, D-Ile4, Arg8, Ala8]vasopressin-(1-8)-OH |
| terlipressin | mozavaptan |
| terlipressin | tolvaptan |
| terlipressin | tolvaptan phosphate ester |
| terlipressin | satavaptan |
| terlipressin | lixivaptan |
| terlipressin | conivaptan |
| terlipressin | RWJ-351647 |
| terlipressin | VP-343 |
| terlipressin | VP-393 |
| terlipressin | [Pmp1, D-Ile4, Arg8, Ala9]vasopressin, |
| terlipressin | [Pmp1, D-Ile4, Arg8, Ala8]vasopressin |
| terlipressin | [Pmp1, D-Ile4, Arg8, Ala8]vasopressin-(1-8)-OH |
| [Gly2]LVP | mozavaptan |
| [Gly2]LVP | tolvaptan |
| [Gly2]LVP | tolvaptan phosphate ester |
| [Gly2]LVP | satavaptan |
| [Gly2]LVP | lixivaptan |
| [Gly2]LVP | conivaptan |
| [Gly2]LVP | RWJ-351647 |
| [Gly2]LVP | VP-343 |
| [Gly2]LVP | VP-393 |
| [Gly2]LVP | [Pmp1, D-Ile4, Arg8, Ala9]vasopressin, |
| [Gly2]LVP | [Pmp1, D-Ile4, Arg8, Ala8]vasopressin |
| [Gly2]LVP | [Pmp1, D-Ile4, Arg8, Ala8]vasopressin-(1-8)-OH |
| [Gly1]LVP | mozavaptan |
| [Gly1]LVP | tolvaptan |
| [Gly1]LVP | tolvaptan phosphate ester |
| [Gly1]LVP | satavaptan |
| [Gly1]LVP | lixivaptan |
| [Gly1]LVP | conivaptan |
| [Gly1]LVP | RWJ-351647 |
| [Gly1]LVP | VP-343 |
| [Gly1]LVP | VP-393 |
| [Gly1]LVP | [Pmp1, D-Ile4, Arg8, Ala9]vasopressin, |
| [Gly1]LVP | [Pmp1, D-Ile4, Arg8, Ala8]vasopressin |
| [Gly1]LVP | [Pmp1, D-Ile4, Arg8, Ala8]vasopressin-(1-8)-OH |
| felypressin | mozavaptan |
| felypressin | tolvaptan |
| felypressin | tolvaptan phosphate ester |
| felypressin | satavaptan |
| felypressin | lixivaptan |
| felypressin | conivaptan |
| felypressin | RWJ-351647 |
| felypressin | VP-343 |
| felypressin | VP-393 |
| felypressin | [Pmp1, D-Ile4, Arg8, Ala9]vasopressin, |
| felypressin | [Pmp1, D-Ile4, Arg8, Ala8]vasopressin |
| felypressin | [Pmp1, D-Ile4, Arg8, Ala8]vasopressin-(1-8)-OH |
| OVP | mozavaptan |
| OVP | tolvaptan |
| OVP | tolvaptan phosphate ester |
| OVP | satavaptan |
| OVP | lixivaptan |
| OVP | conivaptan |
| OVP | RWJ-351647 |
| OVP | VP-343 |
| OVP | VP-393 |
| OVP | [Pmp1, D-Ile4, Arg8, Ala9]vasopressin |
| OVP | [Pmp1, D-Ile4, Arg8, Ala8]vasopressin |
| OVP | [Pmp1, D-Ile4, Arg8, Ala8]vasopressin-(1-8)-OH. |

47. The composition of claim 29, wherein the combination of the vasopressin V2 receptor antagonist with the vasopressin receptor agonist comprises one of the following pairs:

| vasopressin | |
|---|---|
| receptor | |
| agonist: | vasopressin V2 receptor antagonist: |
| AVP | mozavaptan |
| AVP | tolvaptan |
| AVP | tolvaptan phosphate ester |
| AVP | satavaptan |
| AVP | lixivaptan |
| AVP | conivaptan |
| AVP | RWJ-351647 |
| AVP | VP-343 |
| AVP | VP-393 |
| AVP | [Pmp1, D-Ile4, Arg8, Ala9]vasopressin, |
| AVP | [Pmp1, D-Ile4, Arg8, Ala8]vasopressin |
| AVP | [Pmp1, D-Ile4, Arg8, Ala8]vasopressin-(1-8)-OH |
| LVP | mozavaptan |
| LVP | tolvaptan |
| LVP | tolvaptan phosphate ester |
| LVP | satavaptan |
| LVP | lixivaptan |
| LVP | conivaptan |
| LVP | RWJ-351647 |
| LVP | VP-343 |
| LVP | VP-393 |
| LVP | [Pmp1, D-Ile4, Arg8, Ala9]vasopressin, |
| LVP | [Pmp1, D-Ile4, Arg8, Ala8]vasopressin |
| LVP | [Pmp1, D-Ile4, Arg8, Ala8]vasopressin-(1-8)-OH |
| terlipressin | mozavaptan |
| terlipressin | tolvaptan |
| terlipressin | tolvaptan phosphate ester |
| terlipressin | satavaptan |
| terlipressin | lixivaptan |
| terlipressin | conivaptan |
| terlipressin | RWJ-351647 |
| terlipressin | VP-343 |
| terlipressin | VP-393 |
| terlipressin | [Pmp1, D-Ile4, Arg8, Ala9]vasopressin, |
| terlipressin | [Pmp1, D-Ile4, Arg8, Ala8]vasopressin |
| terlipressin | [Pmp1, D-Ile4, Arg8, Ala8]vasopressin-(1-8)-OH |
| [Gly2]LVP | mozavaptan |
| [Gly2]LVP | tolvaptan |
| [Gly2]LVP | tolvaptan phosphate ester |
| [Gly2]LVP | satavaptan |
| [Gly2]LVP | lixivaptan |
| [Gly2]LVP | conivaptan |
| [Gly2]LVP | RWJ-351647 |
| [Gly2]LVP | VP-343 |
| [Gly2]LVP | VP-393 |
| [Gly2]LVP | [Pmp1, D-Ile4, Arg8, Ala9]vasopressin, |
| [Gly2]LVP | [Pmp1, D-Ile4, Arg8, Ala8]vasopressin |
| [Gly2]LVP | [Pmp1, D-Ile4, Arg8, Ala8]vasopressin-(1-8)-OH |
| [Gly1]LVP | mozavaptan |
| [Gly1]LVP | tolvaptan |
| [Gly1]LVP | tolvaptan phosphate ester |
| [Gly1]LVP | satavaptan |
| [Gly1]LVP | lixivaptan |
| [Gly1]LVP | conivaptan |
| [Gly1]LVP | RWJ-351647 |
| [Gly1]LVP | VP-343 |
| [Gly1]LVP | VP-393 |
| [Gly1]LVP | [Pmp1, D-Ile4, Arg8, Ala9]vasopressin, |
| [Gly1]LVP | [Pmp1, D-Ile4, Arg8, Ala8]vasopressin |
| [Gly1]LVP | [Pmp1, D-Ile4, Arg8, Ala8]vasopressin-(1-8)-OH |
| felypressin | mozavaptan |
| felypressin | tolvaptan |
| felypressin | tolvaptan phosphate ester |
| felypressin | satavaptan |
| felypressin | lixivaptan |
| felypressin | conivaptan |
| felypressin | RWJ-351647 |
| felypressin | VP-343 |
| felypressin | VP-393 |
| felypressin | [Pmp1, D-Ile4, Arg8, Ala9]vasopressin, |
| felypressin | [Pmp1, D-Ile4, Arg8, Ala8]vasopressin |
| felypressin | [Pmp1, D-Ile4, Arg8, Ala8]vasopressin-(1-8)-OH |
| OVP | mozavaptan |
| OVP | tolvaptan |
| OVP | tolvaptan phosphate ester |
| OVP | satavaptan |
| OVP | lixivaptan |
| OVP | conivaptan |
| OVP | RWJ-351647 |
| OVP | VP-343 |
| OVP | VP-393 |
| OVP | [Pmp1, D-Ile4, Arg8, Ala9]vasopressin |
| OVP | [Pmp1, D-Ile4, Arg8, Ala8]vasopressin |
| OVP | [Pmp1, D-Ile4, Arg8, Ala8]vasopressin-(1-8)-OH. |

48. The composition of claim 37, wherein the condition comprises:
hypertensive gastropathy bleeding, sepsis, severe sepsis, septic shock, prolonged and severe hypotension, intradialytic hypotension, cardiac arrest, trauma related blood loss, vasodilatory shock induced by cardio-pulmonary bypass, milrinone-induced vasodilatory shock in congestive heart failure, hepatorenal syndrome type I, anaphylactic shock, or cardiovascular instability induced by brain death;
hypotension in severe sepsis, acute respiratory distress syndrome, or acute lung injury; or
inadequate tissue oxygenation, shock induced by metformin intoxication, mitochondrial disease or cyanide poisoning, vascular leak syndrome induced by interleukin-2 or other cytokines, denileukin diftitox or other immunotoxins, or ovarian hyperstimulation syndrome, hypertension induced by end-stage renal disease, severe burns, thermal injury, irritable bowel disease, reperfusion injury, infant respiratory distress syndrome, severe acute respiratory syndrome, ascites, vasodepressor syncope, including vasovagal syncope, postural hypotension with syncope or neurocardiogenic syncope, toxic shock syndrome, idiopathic systemic capillary leak syndrome (Clarkson's disease).

49. The kit of claim 38, wherein the combination of the vasopressin V2 receptor antagonist with the vasopressin receptor agonist comprises one of the following pairs:

| vasopressin | |
|---|---|
| receptor | |
| agonist: | vasopressin V2 receptor antagonist: |
| AVP | mozavaptan |
| AVP | tolvaptan |
| AVP | tolvaptan phosphate ester |
| AVP | satavaptan |
| AVP | lixivaptan |
| AVP | conivaptan |
| AVP | RWJ-351647 |
| AVP | VP-343 |
| AVP | VP-393 |
| AVP | [Pmp1, D-Ile4, Arg8, Ala9]vasopressin, |
| AVP | [Pmp1, D-Ile4, Arg8, Ala8]vasopressin |
| AVP | [Pmp1, D-Ile4, Arg8, Ala8]vasopressin-(1-8)-OH |
| LVP | mozavaptan |
| LVP | tolvaptan |
| LVP | tolvaptan phosphate ester |
| LVP | satavaptan |
| LVP | lixivaptan |
| LVP | conivaptan |
| LVP | RWJ-351647 |
| LVP | VP-343 |
| LVP | VP-393 |
| LVP | [Pmp1, D-Ile4, Arg8, Ala9]vasopressin, |
| LVP | [Pmp1, D-Ile4, Arg8, Ala8]vasopressin |
| LVP | [Pmp1, D-Ile4, Arg8, Ala8]vasopressin-(1-8)-OH |
| terlipressin | mozavaptan |
| terlipressin | tolvaptan |
| terlipressin | tolvaptan phosphate ester |
| terlipressin | satavaptan |
| terlipressin | lixivaptan |
| terlipressin | conivaptan |
| terlipressin | RWJ-351647 |
| terlipressin | VP-343 |
| terlipressin | VP-393 |
| terlipressin | [Pmp1, D-Ile4, Arg8, Ala9]vasopressin, |
| terlipressin | [Pmp1, D-Ile4, Arg8, Ala8]vasopressin |
| terlipressin | [Pmp1, D-Ile4, Arg8, Ala8]vasopressin-(1-8)-OH |
| [Gly2]LVP | mozavaptan |
| [Gly2]LVP | tolvaptan |
| [Gly2]LVP | tolvaptan phosphate ester |
| [Gly2]LVP | satavaptan |
| [Gly2]LVP | lixivaptan |
| [Gly2]LVP | conivaptan |
| [Gly2]LVP | RWJ-351647 |
| [Gly2]LVP | VP-343 |
| [Gly2]LVP | VP-393 |
| [Gly2]LVP | [Pmp1, D-Ile4, Arg8, Ala9]vasopressin, |
| [Gly2]LVP | [Pmp1, D-Ile4, Arg8, Ala8]vasopressin |
| [Gly2]LVP | [Pmp1, D-Ile4, Arg8, Ala8]vasopressin-(1-8)-OH |
| [Gly1]LVP | mozavaptan |
| [Gly1]LVP | tolvaptan |
| [Gly1]LVP | tolvaptan phosphate ester |
| [Gly1]LVP | satavaptan |
| [Gly1]LVP | lixivaptan |
| [Gly1]LVP | conivaptan |
| [Gly1]LVP | RWJ-351647 |
| [Gly1]LVP | VP-343 |
| [Gly1]LVP | VP-393 |
| [Gly1]LVP | [Pmp1, D-Ile4, Arg8, Ala9]vasopressin, |
| [Gly1]LVP | [Pmp1, D-Ile4, Arg8, Ala8]vasopressin |
| [Gly1]LVP | [Pmp1, D-Ile4, Arg8, Ala8]vasopressin-(1-8)-OH |
| felypressin | mozavaptan |
| felypressin | tolvaptan |
| felypressin | tolvaptan phosphate ester |
| felypressin | satavaptan |
| felypressin | lixivaptan |
| felypressin | conivaptan |
| felypressin | RWJ-351647 |
| felypressin | VP-343 |
| felypressin | VP-393 |
| felypressin | [Pmp1, D-Ile4, Arg8, Ala9]vasopressin, |
| felypressin | [Pmp1, D-Ile4, Arg8, Ala8]vasopressin |
| felypressin | [Pmp1, D-Ile4, Arg8, Ala8]vasopressin-(1-8)-OH |
| OVP | mozavaptan |
| OVP | tolvaptan |
| OVP | tolvaptan phosphate ester |
| OVP | satavaptan |
| OVP | lixivaptan |
| OVP | conivaptan |
| OVP | RWJ-351647 |
| OVP | VP-343 |
| OVP | VP-393 |
| OVP | [Pmp1, D-Ile4, Arg8, Ala9]vasopressin |
| OVP | [Pmp1, D-Ile4, Arg8, Ala8]vasopressin |
| OVP | [Pmp1, D-Ile4, Arg8, Ala8]vasopressin-(1-8)-OH. |
